(19) United States
(12) Patent Application Publication
(10) Pub. No.: US 2023/0086256 A1
(43) Pub. Date: Mar. 23, 2023

(54) DRUG DELIVERY SYSTEM FOR THE DELIVERY OF STEROID TO VITREOUS CHAMBER OF THE EYE

(71) Applicants: Morgan B. Giles, Rahway, NJ (US); Seth P. Forster, West Point, PA (US); Stephanie Elizabeth Barrett, Rahway, NJ (US); Steven C. Persak, Rahway, NJ (US); Merck Sharp & Dohme LLC, Rahway, NJ (US)

(72) Inventors: Morgan B. Giles, Weehawken, NJ (US); Seth P. Forster, Fort Washington, PA (US); Stephanie Elizabeth Barrett, Cranford, NJ (US); Steven C. Persak, Morris Plains, NJ (US)

(73) Assignee: Merck Sharp & Dohme LLC, Rahway, NJ (US)

(21) Appl. No.: 17/793,583

(22) PCT Filed: Jan. 22, 2021
(86) PCT No.: PCT/US2021/014491

§ 371 Date: Jul. 18, 2022

Related U.S. Application Data
(60) Provisional application No. 62/966,699, filed on Jan. 28, 2020.

(51) Int. Cl. A61F 9/00; A61K 9/00

(52) U.S. Cl. CPC A61F 9/0017; A61K 9/0051; A61P 27/02

(57) ABSTRACT

This invention relates to novel implant drug delivery systems for long-acting delivery of mometasone furoate. These compositions are useful for the treatment or prevention of inflammatory conditions of the eye.

### BACKGROUND OF THE INVENTION

Anti-inflammatory agents are useful for the treatment of inflammatory conditions of the eye. Topical drug administration (i.e. eye drops) has proven successful for treatment of anterior ocular diseases as it is non-invasive, easy to administer and has high patient compliance. However, topical administration for treatment of posterior eye diseases suffer from poor bioavailability (less than 5%) due to barriers of the anterior segment of the eye, such as the corneal epithelium and conjunctiva. Posterior eye diseases, such as macular edema and diabetic retinopathy, are therefore most commonly treated via intravitreal injection. Intravitreal injections are highly invasive and can require repeated injections due to the short half-life of drugs in the vitreous, therefore risking potential retinal detachment, hemorrhage, endophthalmitis and poor patient tolerance. Intravitreal injections of biodurable or bioerodible polymer implants allow for localized drug delivery over extended periods of time, thus reducing the need for repeated injections and potentially improving patient tolerance.

Thus, novel formulation approaches capable of delivering extended-duration pharmacokinetic characteristics for molecules to treat inflammatory conditions of the eye, in particular posterior eye diseases, are highly desirable.

Mometasone furoate is a corticosteroid drug that can be used for the treatment of asthma, rhinitis, nasal polyps and certain skin conditions. It has a glucocorticoid receptor binding affinity 22 times stronger than dexamethasone and higher than many other corticosteroids as well, see, Hubner M, Hochhaus G, Derendorf H: Comparative pharmacology, bioavailability, pharmacokinetics, and pharmacodynamics of inhaled glucocorticosteroids. Immunol Allergy Clin North Am. 2005 Aug; 25(3):469-88. Mometasone furoate is formulated as a dry powder inhaler, nasal spray, sinus implant and ointment for its different indications.

The instant invention provides novel formulation approaches capable of delivering mometasone furoate to treat inflammatory conditions of the eye.

### SUMMARY OF THE INVENTION

This invention relates to novel implant drug delivery systems for long-acting delivery of mometasone furoate into the vitreous of the eye. These compositions are useful for the treatment or prevention of inflammatory conditions of the eye, in particular posterior eye diseases.

### DETAILED DESCRIPTION OF THE INVENTION

This invention relates to novel implant drug delivery systems for long-acting delivery of mometasone furoate into the vitreous of the eye. The novel implant drug delivery systems comprise a biocompatible bioerodible polymer and mometasone furoate. These implant drug delivery systems are useful for the treatment or prevention of inflammatory conditions of the eye. The invention further relates to methods of treating and preventing inflammatory conditions of the eye with the novel implant drug delivery systems described herein.

As used herein, “inflammatory conditions of the eye” includes, but is not limited to, uveitis, posterior uveitis, macular edema, acute macular degeneration, wet age related macular edema, retinal detachments, retinal vein occlusion, ocular tumors, fungal infections, viral infections, multifocal choroiditis, diabetic uveitis, diabetic macular edema, diabetic retinopathy, proliferative vitreoretinopathy, sympathetic ophthalmia, Vogt Koyanagi-Harada syndrome, histoplasmosis and uveal diffusion.

As used herein, “posterior eye disease” includes, but is not limited to, uveitis, posterior uveitis, wet age related macular edema, diabetic macular edema, diabetic retinopathy and retinal vein occlusion.

The novel implant delivery systems of the invention comprise a biocompatible bioerodible polymer to generate monolithic matrices with dispersed or dissolved drug. The chemical properties of the polymer matrices are tuned to achieve a range of drug release characteristics, offering the opportunity to extend duration of dosing. In an embodiment of the invention, the novel implant delivery systems are compatible with molecules having a broad spectrum of physicochemical properties, including those of high aqueous solubility or amorphous phases which are unsuitable to formulation as solid drug suspensions.

Specifically, this invention relates to an implant drug delivery system comprising an implant, which comprises a core which comprises:
(a) a biocompatible bioerodible polymer and
(b) mometasone furoate, which is present in the core between 5% to 70% by weight,
   wherein said implant is implanted into the vitreous of the eye and mometasone furoate is continually released into the vitreous at a rate resulting in a plasma concentration of mometasone furoate in ocular tissue between 0.1 and 0.5 mcg/d for a period of three months to thirty-six months. These implant delivery systems are desired and useful for prophylaxis and/or treatment of inflammatory conditions of the eye from both compliance and convenience standpoints.

As used herein, the term “biocompatible bioerodible polymer” refers to polymeric materials that include hydrolytically labile linkages which undergo cleavage at physiological conditions. The broken down products are non-toxic and either excreted in the urine or incorporated into the Krebs cycle and used for energy. The polymer is generally hydrophobic so that it retains its integrity for a suitable period of time when placed in an aqueous environment, such as the body of a mammal, and is stable enough to be stored for an extended period before use. Bioerodible polymers remain intact in vivo for extended periods of time, typically weeks, months or years. Drug molecules encapsulated in the polymer are released over time via diffusion through channels and pores in a sustained manner. The release rate can be altered by modifying the identity of the polymer (monomeric units, molecular weight, end group, etc.) thereby modifying the degradation kinetics, percent drug loading, porosity of the polymer, structure of the implantable device, or hydrophobicity of the polymer, or by adding a hydrophobic coating to the exterior of the implantable device.

Accordingly, any polymer that can be readily cleared or eliminated by the body can be used to manufacture the implant drug delivery systems of the instant invention. The term “polymer” can also include copolymers. Biocompatible bioerodible polymers of the instant invention include, but are not limited to, poly(DL-lactide) (“PLA”), poly(caprolactone) (“PCL”), poly(lactide-co-glycolide), poly(lactide), poly(glycolide) (“PLG”), poly(ortho esters), poly(dioxanone), poly(alkylcyanoacrylates) and combinations thereof. In a class of the invention, the biocompatible bioerodible polymer is poly(caprolactone).

In a class of the invention, the biocompatible bioerodible polymer is selected from the group consisting of poly(DL-lactide), poly(L-lactide), and poly(caprolactone), all of which can have an acid or an ester end group. In a subclass of the invention, the biocompatible bioerodible polymer is poly(caprolactone).

As used herein, the term “diffusional barrier” refers to a coating that is permeable to the drug and is placed over at least a portion of the device to further regulate the rate of release. For example, a coating of biocompatible bioerodible polymeric material, e.g., poly(DL-lactide), or a coating of a biocompatible bioerodible polymeric material with a lower drug loading than the remainder of the implant delivery system, may be used. The diffusional barrier may be formed, for example, by coextrusion with the device.

Suitable diffusional barriers of the instant invention include, but are not limited to, poly(DL-lactide) (“PLA”), poly(caprolactone) (“PCL”), poly(lactide-co-glycolide), poly(lactide), poly(glycolide), poly(ortho esters), poly(dioxanone), poly(alkylcyanoacrylates) and combinations thereof. In a class of the invention, the diffusional barrier is selected from the group consisting of poly(DL-lactide) and poly(caprolactone).

In an embodiment of the invention, the diffusion barrier contains mometasone furoate.

As used herein, the term “dispersed or dissolved in the biocompatible bioerodible polymer” refers to the drug and polymer being mixed and then hot-melt extruded or injection molded.

As used herein, the term “continually released” refers to the drug being released from the biocompatible bioerodible polymer at continuous rates for extended periods of time. The implant drug delivery systems of the instant invention generally exhibit 1^{st} order release kinetics for the drug in vivo, sometimes with an initial burst. Polymer degradation modifies the dissolution and diffusion of the drug from the polymer matrix. This typically results in an increased drug elution rate that may deviate from first order kinetics, and is a function of the polymer degradation rate.

Optionally, the novel implant delivery systems of the instant invention can further comprise a radiopaque component. The radiopaque component will cause the implant to be X-ray visible. The radiopaque component can be any such element known in the art, such as barium sulfate, titanium dioxide, bismuth oxide, tantalum, tungsten or platinum. In a specific embodiment, the radiopaque component is barium sulfate.

In one embodiment, the radiopaque material is 1% to 30% by weight. In another embodiment, the radiopaque material is 1% to 20% by weight. In another embodiment, the radiopaque material is 4% to 25% by weight. In further embodiment, the radiopaque material is 6% to 20% by weight. In another embodiment, the radiopaque material is 4% to 15% by weight. In another embodiment, the radiopaque material is about 8% to 15% by weight.

The radiopaque material does not affect the release of mometasone furoate from the implant.

The novel implants of the instant invention can be sterilized. In an embodiment of the invention, the implant is sterilized by gamma irradiation. In a class of the embodiment, the gamma irradiation dose is 25-50 k Gy. In a subclass of the invention, the gamma irradiation dose is 25-40 k Gy. In a further subclass of the invention, the gamma irradiation dose is 25.5-26.8 k Gy. In another embodiment of the invention, the implant delivery system is sterilized by E-beam sterilization. In another embodiment of the invention, the implant delivery system is sterilized by x-ray sterilization.

The novel implant delivery systems of the invention comprise mometasone furoate. In an embodiment of the invention, mometasone furoate is administered as a monotherapy. In another embodiment of the invention mometasone furoate is administered in combination with another agent, including but not limited to an anti-inflammatory agent, anti-VEGF agent and/or an immunosuppressant agent.

An “anti-inflammatory agent” is any agent which is directly or indirectly effective in the reduction of inflammation when administered at a therapeutically effective level. “Anti-inflammatory agent” includes, but is not limited to steroidal anti-inflammatory agents and glucocorticoids. Suitable anti-inflammatory agents include, but are not limited to, cortisone, dexamethasone, hydrocortisone, methylprednisolone, prednisolone, prednisone and triamcinolone.

An “anti-VEGF agent” is any agent which is directly or indirectly effective in inhibiting the activity of VEGF (Vascular Endothelial Growth Factor). Suitable anti-VEGF agents include, but are not limited to, bevacizumab, ranibizumab and aflibercept.

In an embodiment of the invention mometasone furoate is administered in combination with bevacizumab. In another embodiment of the invention mometasone furoate is administered in combination with ranibizumab. In another embodiment of the invention mometasone furoate is administered in combination with aflibercept.

In an embodiment of the invention the implant drug delivery system comprises mometasone furoate and bevacizumab. In another embodiment of the invention the implant drug delivery system comprises mometasone furoate and ranibizumab. In another embodiment of the invention the implant drug delivery system comprises mometasone furoate and aflibercept.

An “immunosuppressant agent” is any agent which is directly or indirectly effective in suppressing, or reducing, the strength of the body's immune system. Suitable immunosuppressant agents include, but are not limited to, corticosteroids (for example, prednisone, budesonide, prednisolone), janus kinase inhibitors (for example, tofacitinib), calcineurin inhibitors (for example, cyclosporin, tacrolimus), mTOR inhibitors (for example, sirolimus, everolimus), IMDH inhibitors (for example, azathioprine, leflunomide, mycophenolate), biologics (for example, abatacept, adalimumab, anakinra, certolizumab, etanercept, golimumab, infliximab, ixekizumab, natalizumab, rituximab, secukinumab, tocilizumab, ustekinumab, vedolizumab), and monoclonal antibodies (for example, basiliximab, daclizumab).

In certain embodiments the anti-inflammatory agents, anti-VEGF agents and immunosuppressant agents described herein are employed in their conventional dosage ranges and regimens as reported in the art, including, for example, the dosages described in editions of the Physicians' Desk Reference, such as the 70th edition (2016) and earlier editions. In other embodiments, the anti-inflammatory agents, anti-VEGF agents and immunosuppressant agents described herein are employed in lower than their conventional dosage ranges.

Mometasone furoate (“MF”) has the chemical name 9,21-Dichloro-11β,17-dihydroxy-16α-methylpregna-1,4-diene-3,20-dione17-(2 furoate), and has the following chemical structure:

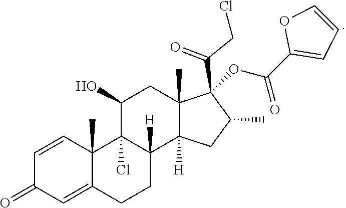

In an embodiment of the implant drug delivery system described herein, the mometasone furoate is present in the core at 5% - 70% by weight of drug loading. In other embodiments, the mometasone furoate is present in the core at 20%-70% by weight of drug loading, at 20%-60% by weight of drug loading or at about 40%-60% by weight of drug loading. In an example of the embodiment of the implant drug delivery system described herein, the mometasone furoate is present in the core at 20% by weight of drug loading. In another example of the embodiment of the implant drug delivery system described herein, the mometasone furoate is present in the biocompatible bioerodible polymer at 60% by weight of drug loading.

The implant drug delivery systems of the instant invention may be produced by injection molding. Alternatively, the implant drug delivery systems of the instant invention may be produced using an extrusion process, wherein ground biocompatible, bioerodible polymer or biocompatible, bioerodible polymer pellets are blended with the corticosteroid, melted and extruded into rod-shaped structures. Rods are cut into individual implantable devices of the desired length, packaged and sterilized prior to use. Other methods for encapsulating therapeutic compounds in implantable polymeric, bioerodible matrices are known to those of skill in the art. Such methods include solvent casting (see U.S. Pat. Nos. 4,883,666, 5,114,719 and 5,601,835). One of skill in the art would be able to readily determine an appropriate method of preparing such an implant drug delivery system, depending on the shape, size, drug loading, and release kinetics desired for a particular type of patient or clinical application.

The size and shape of the implant drug delivery systems may be modified to achieve a desired overall dosage. The implant drug delivery systems of the instant invention are often about 0.2 mm to about 10 mm in length. In an embodiment of the invention, the implant drug delivery systems are about 0.2 mm to about 3 mm in length. In a class of the embodiment, the implant drug delivery systems are about 5 mm to about 10 mm in length. In a subclass of the embodiment, the implant drug delivery systems are about 5 mm in length. The implant drug delivery systems of the instant invention are often about 0.1 mm to about 2 mm in diameter. In an embodiment of the invention, the implant drug delivery systems are about 0.25 mm to about 0.5 mm in diameter. In a class of the embodiment, the implant drug delivery systems are about 0.25 mm in diameter. In another class of the embodiment, the implant drug delivery systems are about 0.5 mm in diameter.

The implant drug delivery systems described herein are capable of releasing mometasone furoate over a period of 21 days, 28 days, 31 days, 4 weeks, 6 weeks, 8 weeks, 12 weeks, one month, two months, three months, four months, five months, six months, seven months, eight months, nine months, ten months, eleven months, twelve months, eighteen months, twenty-four months or thirty-six months at an average rate of between 0.01-0.5 mcg per day. In an embodiment of the invention, the implant drug delivery systems described herein are capable of releasing mometasone furoate at a rate of between 0.01-5 mcg per day. In an embodiment of the invention, the mometasone furoate is released at therapeutic concentrations for a duration from between three months and thirty-six months. In a class of the embodiment, the mometasone furoate is released at therapeutic concentrations for a duration from between six months and twelve months.

One or more implants can be used to achieve the desired therapeutic dose. In an embodiment of the invention, one or more implants can be used to achieve the therapeutic dose for durations of up to 1 year. In another embodiment of the invention, one or more implants can be used to achieve the therapeutic dose for durations of up to 2 years.

The implant drug delivery systems described herein are capable of releasing mometasone furoate resulting in a plasma concentration of mometasone furoate in ocular tissue between 0.1-0.5 mcg per day. In an embodiment of the invention, the implant drug delivery systems described herein are capable of releasing mometasone furoate resulting in a plasma concentration of mometasone furoate in ocular tissue between 0.1-0.3 mcg per day. In another embodiment of the invention, the implant drug delivery systems described herein are capable of releasing mometasone furoate resulting in a plasma concentration of mometasone furoate in ocular tissue between 0.2-0.4 mcg per day.

The following examples are given for the purpose of illustrating the present invention and shall not be construed as being limitations on the scope of the invention.

### EXAMPLE 1

### Preparation of Implant Drug Delivery Systems Containing 20-60 Wt % Mometasone Furoate

Implants were prepared using an injection molding process. Milled Polycaprolactone (PCL) and Mometasone Furoate were blended with 20 wt % and 60 wt % drug. This blend was heated during blending in a high-speed rotary mixer or melt extruded with a twin screw extruder at temperatures ranging from 60° C.-100° C., screw speed at 20-50 rpm, and then granulated or pelletized. The pellets were then placed in an injection molder with the feed barrel set to 60° C. (actual temperature ranges from 60° C.-80° C.) with mold temperature ranges set between 30° C.-38° C. (actual temperature ranges from 29° C.-42° C.). The mold was designed with multiple radial 0.25 mm channels. The part is then removed and the implants were cut to 5.0±0.1 mm length. The implants were gamma irradiated to sterilize.

The in vitro release rate of mometasone furoate was determined using a manual dissolution technique. The full implant was put into a microcentrifuge tube and was submerged in 1 mL of phosphate buffered saline (PBS)+sodium dodecyl sulfate (SDS). A temperature of 37° C. was maintained in an incubator. Samples were gently shaken by the system. The volume of PBS was sufficient to maintain sink conditions. Sink conditions are defined as the drug concentration maintained at or below 1/3 of the maximum solubility (drug concentration ≤0.45 mg/mL in PBS at 37° C.). A 1 mL sample was removed once per day and filled it into an HPLC vial. A full media (1 mL) replacement was performed every day (24 h) for the first week followed by weekly replacement thereafter. Samples were assayed by HPLC (Waters Alliance 2695). Analysis of a 15 μL volume was performed at 254 nm with an XTerra RP18 column (150×4.6 mm, 3.5 μm). The mobile phase was water and 50:50 MeOH:water (45:55 v/v) at a flow rate of 1 mL/min.

To determine degradation of Mometasone furoate, a 6 μL volume was injected onto a Water Atlantis T3 column (150×4.6 mm, 3 μm) maintained at 40° C. The mobile phase was 0.1% TFA (trifluoroacetic acid) in Water and 0.1% TFA in 50:50 (v/v) ACN:MeOH (acetonitrile:methanol) with a flow rate of 1.5 mL/min. The mobile phase gradient is shown in the table below.

TABLE 1
Mometasone furoate
chemical stability
HPLC method details

| | 0.1 |
|---|---|
| Time | TFA in |
| (min) | Water |
| 0.0 | 100 |
| 15.0 | 92 |
| 30.0 | 70 |
| 40.0 | 10 |
| 40.1 | 100 |
| 45.0 | 100 |

TABLE 2
Mometasone furoate (“MF”) in vitro release
from 20 wt % and 60 wt % Mometasone
furoate in Poly(caprolactone) (“PCL”)
implants; reported as a % release from total
[avg = average and std dev = standard deviation]

| | 20 wt % | | 60 wt % | |
|---|---|---|---|---|
| | MF in PCL | | MF in PCL | |
| Time | | std. | | std. |
| (days) | avg (%) | dev | avg (%) | dev |
| 1 | 0.7 | 0.3 | 0.3 | 0.2 |
| 2 | 2.1 | 1.4 | 0.5 | 0.2 |
| 3 | 3.0 | 1.7 | 1.0 | 0.4 |
| 4 | 4.1 | 2.1 | 1.4 | 0.3 |
| 7 | 5.6 | 2.0 | 1.9 | 0.5 |
| 14 | 8.6 | 2.3 | 2.7 | 0.8 |
| 21 | 11.2 | 2.5 | 3.5 | 1.0 |
| 28 | 14.3 | 2.9 | 4.5 | 1.4 |
| 35 | 17.2 | 3.2 | 5.6 | 1.8 |
| 42 | 21.0 | 4.6 | 6.9 | 2.2 |
| 50 | 25.1 | 6.2 | 8.3 | 2.8 |
| 56 | 28.8 | 6.8 | 9.3 | 3.0 |
| 63 | 31.6 | 7.6 | 10.2 | 3.3 |
| 70 | 35.0 | 8.9 | 11.3 | 3.8 |
| 84 | 39.0 | 9.5 | 12.6 | 4.1 |
| 98 | 42.9 | 9.7 | 14.1 | 4.4 |
| 140 | 47.8 | 9.7 | 16.0 | 4.7 |
| 157 | 51.7 | 9.7 | 17.7 | 5.1 |
| 169 | 55.1 | 9.6 | 19.4 | 5.4 |

What is claimed is:

1. An implant drug delivery system comprising an implant, which comprises a core which comprises:
(a) a biocompatible bioerodible polymer and
(b) mometasone furoate, which is present in the core between 5% to 70% by weight,
wherein said implant is implanted into the vitreous of the eye and mometasone furoate is continually released into the vitreous at a rate resulting in a plasma concentration of mometasone furoate in ocular tissue between 0.1 and 0.5 mcg/d for a period of three months to thirty-six months.

2. The implant drug delivery system of claim 1 wherein the plasma concentration of mometasone furoate in ocular tissue is between 0.1 mcg/d and 0.3 mcg/d.

3. The implant drug delivery system of claim 2 wherein the plasma concentration of mometasone furoate in ocular tissue is between 0.2 mcg/d and 0.4 mcg/d.

4. The implant drug delivery system of claim 1 wherein the mometasone furoate is present in the core between 20% to 70% by weight.

5. The implant drug delivery system of claim 4 wherein the mometasone furoate is present in the core between 20% to 60% by weight.

6. The implant drug delivery system of claim 5 wherein the mometasone furoate is present in the core between 40% to 60% by weight.

7. The implant drug delivery system of claim 5 wherein the mometasone furoate is present in the core at about 20% by weight.

8. The implant drug delivery system of claim 5 wherein the mometasone furoate is present in the core at about 60% by weight

9. The implant drug delivery system of claim 1 wherein the biocompatible bioerodible polymer is selected from the group consisting of poly(DL-lactide), poly(caprolactone), poly(lactide-co-glycolide), poly(lactide), poly(glycolide), poly(ortho esters), poly(dioxanone), poly(alkylcyanoacrylates) and combinations thereof

10. The implant drug delivery system of claim 9 wherein the biocompatible bioerodible polymer is poly(caprolactone).

11. The implant drug delivery system of claim 1 further comprising between 1% and 20% by weight of a radiopaque material.

12. The implant drug delivery system of claim 1 wherein the implant is sterilized.

13. The implant drug delivery system of claim 12 wherein the implant is sterilized by gamma irradiation.

14. The implant drug delivery system of claim 13 wherein the implant is sterilized at a gamma irradiation dose of between 20-50 k Gy.

15. The implant drug delivery system of claim 1 wherein the mometasone furoate is released at therapeutic concentrations for a duration from between three months and twelve months.

16. A method of treating an inflammatory condition of the eye with an implant drug delivery system according to claim 1.

17. The method of claim 1 wherein the inflammatory condition of the eye is selected from the group consisting of uveitis, posterior uveitis, macular edema, acute macular degeneration, retinal detachments, retinal vein occlusion, ocular tumors, fungal infections, viral infections, multifocal choroiditis, diabetic uveitis, diabetic macular edema, proliferative vitreoretinopathy, sympathetic ophthalmia, Vogt Koyanagi-Harada syndrome, histoplasmosis and uveal diffusion.

18. The method of claim 17 wherein the inflammatory condition of the eye is diabetic macular edema.

19. The method of claim 17 wherein the inflammatory condition of the eye is posterior uveitis.

20. The method of claim 17 wherein the inflammatory condition of the eye is retinal vein occlusion.
